(19) United States
(12) Patent Application Publication
(10) Pub. No.: US 2006/0089545 A1
(43) Pub. Date: Apr. 27, 2006

(54) System and method for presenting and distributing medication information

(75) Inventors: Jochen Ratjen, Nacka (SE); Lennart Brunnberg, Tyreso (SE)

Correspondence Address: NIXON & VANDERHYE, PC, 901 NORTH GLEBE ROAD, 11TH FLOOR, ARLINGTON, VA, 22203, US

(73) Assignee: SHL MEDICAL AB, Nacka Strand (SE)

(21) Appl. No.: 10/549,328

(22) PCT Filed: Feb. 25, 2004
(86) PCT No.: PCT/SE04/00251

§ 371 Date: Oct. 24, 2005

(30) Foreign Application Priority Data
Mar. 18, 2003 (SE) 0300729-1

(51) Int. Cl. A61B 5/00

(52) U.S. Cl. 600/309; 604/890.1

(57) ABSTRACT

The present invention relates generally to a system for presenting and distributing information related to the medication of a patient comprising a terminal device and a method of presenting and distributing information related to the medication of the patient in such a system. The system comprises a terminal device communicating with a wireless communication system, and a medicament device including medicament containing means arranged to hold a medicament; a dispensing mechanismarranged for dispensing medicament from the medicament containing means; means for obtaining information related to the medication of the patient; and a communication device for wireless communication to enable transfer of data related to the information from the medicament device to the terminal device comprising, inter alia, means for receiving the data from the communication device and being arranged to present information related to the medication of the patient to a user.

### TECHNICAL FIELD

The present invention relates generally to a system for presenting and distributing information related to the medication of a patient comprising a terminal device and a method of presenting and distributing information related to the medication of the patient in such a system. The invention further relates to a computer readable medium comprising instructions for bringing a computer or programmable device to perform such a method.

### BACKGROUND OF THE INVENTION

Each year, numerous patients are admitted to bad conditions and/or to hospitals for complications resulting from non-compliance. Among the most frequently cited reasons for non-compliance are failing to take the proper medication or combinations of medications, administrating the incorrect dosage, or forgetting to take the proper medication altogether. For certain minor illnesses, failing to take the medication may result in mild discomfort that may be treated on an outpatient basis. For more serious illnesses, however, medication non-compliance can result in severe by-effects and/or illness.

In addition, failure to follow a prescribed treatment ultimately may make the virus or bacteria resistant to treatment and create a potential health risk by creating drug-resistant strains of the disease.

One reason of medication non-compliance is that the patient forgets or misreads the instructions for patient administration of the medicament according to a defined treatment regime. Normally, a doctor prescribes a patient with medicament in a medicament device together with instructions for patient administration of the medicament according to the defined treatment regime. The patient typically therefore, receives instructions relating to the correct use of the device together with the recommended dosing amounts, dose intervals and treatment period. The patient is then trusted to follow the treatment regime as set by the doctor.

However, various systems have been proposed to address the problem of patient medication non-compliance.

WO 01/24690 describes a system for the delivery of medicament comprising a dose inhaler having an electronic data management system and a communicator for wireless communication with network computer system to enable communication of data between the network computer system and the electronic data management system. The electronic data management system comprises a memory for storage of data and a microprocessor for performing operations on the data. A cellular phone may be used as transmission link for the transmission of data between the communicator and the network computer system using spread spectrum radiofrequency signals. The system can feed information, such as compliance information, to the network computer system. Furthermore, the patient can also communicate requests for data to the network and receive responses via a transmission link. For that purpose, the inhaler comprises push-buttons and a display for allowing a display of menu choices and data from the electronic data management system or the network.

However, the system described in WO 01/24690 has several drawbacks. For example, the inhaler is expensive due to the complex design including, inter alia, the display for the display of menu choices and data from the electronic data management system and the integrated circuit in which the electronic data management system is implemented. This also entails to the high manufacturing costs of the inhaler. Therefore, the price of the inhaler will inevitably be high. The development costs for the software necessary to, for example, obtain the interfacing between the different components of the inhaler, handle the menus and the data from the electronic data management system, and the electronic data management system itself are significantly high, a fact that also contributes to the high price of the inhaler and the associated system.

Another problem is the applicability of the inhaler. A common user-requirement is that the inhaler should be small since it must be easy to carry and not too visible. On the other hand, an inhaler having a limited size necessarily limits the space available for the display and the push-buttons and the like, which, in turn, limits the functionality of the inhaler. Accordingly, there is an inherent contradiction between the size of the inhaler and the functionality of the inhaler, i.e. the services that the inhaler is able to provide in terms of medicament information.

Thus, there is a problem to find a system that provides a solution to the medication compliance problem at a low cost. In particular, there is a problem to find a system capable of delivering medicaments that present a high functionality, for instance a function for reminding the patient to comply with medication requirements, and is user-friendly, and that, in the same time, incorporates a simple and inexpensive medicament device.

Further, there is a problem to find a system that is able to provide the patient with treatment instructions that are easy to follow for the patient.

Furthermore, there is a need for a system that in an effective way, both in respect of user convenience and economic aspects, monitors the delivery of medicament.

Further, a need exists for a system that monitors medication compliance.

Further, a need exists for a system that can hold many different types of medication.

### SUMMARY OF THE INVENTION

One object of the present invention is to provide a solution to the problem discussed above. This and other objects are obtained according to the present invention by providing a system, a method and a computer readable medium having the features defined in the independent claims. Preferred embodiments are defined in the dependent claims.

For purposes of clarity the term proper medication can refer to, inter alia, strength, amount (or dose), or kind of medication, as well as time between doses, maximum or minimum time between doses.

According to a first aspect of the present invention, there is provided a system for presenting and distributing information related to a medication of a patient comprising a terminal device communicating with a wireless communication system, and a medicament device including a medicament containing means arranged to hold a medicament; a dispensing mechanism arranged for dispensing medicament from the medicament containing means; means for obtaining information related to the medication of a patient; and a communication device for wireless communication to enable transfer of data related to the information from the medicament device to a terminal device comprising, inter alia, means for receiving the data from the communication device and being arranged to present information related to the medication of the patient to a user.

According to a second aspect of the invention, there is provided a method of presenting information related to a medication of a patient in a system comprising a terminal device communicating with a wireless communication system, and a medicament device including a medicament containing means arranged to hold a medicament; a dispensing mechanism arranged for dispensing medicament from the medicament containing means; means for obtaining information related to the medication of a patient; and a communication device for wireless communication to enable transfer of data related to the information from the medicament device to a terminal device. The method comprising the step of utilizing the terminal device to present information related to the medication of the patient to a user.

According to a further aspect of the invention there is provided a computer readable medium comprising instructions for bringing a programmable device to perform the method according the second aspect of the invention.

Thus, the present invention is based on the idea of using the functionality of a terminal device such as a cellular or a mobile phone or a PDA, i.e. a palmtop, a palm pilot, or the like, to present, in a system for presenting and distributing information related to a medication of a patient, information related to the medication of a patient to a user of the terminal device. By utilizing the in-built functionality of a terminal device, for example, the display and the key-set or key-pad to provide the user with medication information, the medicament device in such a system, e.g. an inhaler, an injector device, a tablet dispenser, or the like, can be made small and cheap. This is owing to the fact that instead of incorporating the components necessary to provide medication compliance information functions in the medicament device, the in-built components of a terminal device, for instance a cellular phone, are used for presenting information of different types.

This solution provides several advantages over the existing solutions. One advantage is that the presentation of the medication information can be made more perceivable for a user, for example the patient. This significantly reduces the risks of medication non-compliance, which is an evident risk with the inhaler disclosed in WO 01/24690 where the size of the display is limited and the quality may be inferior and, thereby, the patient may misread the instructions for patient administration of the medicament according to a defined treatment regime.

Accordingly, the present invention is suitable for use in a number of areas related to the medication compliance, for example, contraception supervision, allergy medication, medication related to cardiac diseases, medication related to asthma, medication related to COPD (chronic obstructive pulmonary disease), pain relief, mental illnesses or other time critical medication regimes, as well as at clinical studies where the results often depend completely or to a large extent on the ability of the participants of the study to comply with the time schedule for taking the medication.

Another advantage is that the medicament device can be made at a low cost per unit in comparison to the device disclosed in WO 01/24690 due to the fact that a significant part of the functionality of the medicament device has been transferred to a terminal device.

Furthermore, the medicament device included in the system offers high degree of applicability to the patient since it can be made small and, at the same time, provide user-friendly functions for medical compliance, monitoring, or supervision.

According to preferred embodiments of the present invention, medication information is obtained from the medicament device by the means for obtaining information related to the medication, which information may be, inter alia, dosage information or timing information. Subsequently, the information is transferred to a terminal device from the medicament device via the communication device. Thereby, it is possible to present medical compliance information to the user, e.g. the patient, of the occurrence or absence of medication events related to the medicament device using the terminal device.

According to the present invention, the user is notified of an event related to the medication by using the functionality of a terminal device, e.g. a cellular phone. Thereby, it is possible to, for example, remind the user that he or she has forgotten to take the medication or that he or she has not taken the proper medication, or the proper medication in time.

According to preferred embodiments, the user is notified of the occurrence of an event related to the medication through means for notifying selected from the group consisting of means for providing audible signals, visualizing means, vibration means, and light means.

The means for proving audible signals may be, for example, a speaker phone of a cellular phone, which is able to notify the user by producing an audible signal. In order to increase the medication compliance a specific signal, for example, a characteristic tune, may be selected. Thereby, the user is able to directly recognize the signal. The visualizing means may be, for example, a display comprising a screen such as an LED or LCD. If a cellular phone is utilized the user may be notified, for example, by an evident symbol, or a message, or an instructive sequence of pictures, or a film sequence shown on the display. If a cellular phone is used the vibration means may be, for example, a vibration unit. The user is notified by a vibration of the unit. This is advantageous in noisy environments such as a subway or in a restaurant. The light means may be, for example, a diode or the display of a cellular phone.

Due to the fact that the functionality of the terminal device, for example, a cellular phone is utilized to present different types of information in distinct ways, the notifying is facilitated and efficient, which significantly reduces the risk of medication non-compliance. Furthermore, the notification or signal given by the terminal device can be set to minimize the risk of embarrassment of the user, for example, by utilizing a discreet notification that do not attracts public attention. Moreover, since the functionality for notifying the patient has been transferred or transmitted to the terminal device from the medicament device, the construction of the device is simple and, thereby, the device can be manufactured at a low cost. In addition the medicament device can be made in a small size, which increases the portability.

Furthermore, the user is notified by means of, for example, an SMS message, an MMS message, or the like.

In a specific embodiment, the notification of the occurrence of an event related to the medication comprises a first signal or notification and a number of subsequent reminder signals, which may be in the same form as the first notification or in an other form. This further reduces the risk of medication non-compliance.

Furthermore, the medicament device can be arranged to send status indicating messages to a terminal device. Such a status indicating message can, for example, indicate that the battery capacity of the medicament device is running out or the medical substance contained in the medicament device is about to run out. In case of a battery capacity warning the message may be sent when the capacity has reached a certain predetermined level, thus giving the user of the terminal device a warning in advance, and, in the same way, a labelled device life can be indicated for the user, a doctor, or a service provider.

According to other embodiments, the communication device of the medicament device is arranged for two-way transfer of data between a terminal device and the medicament device. Thereby, medication data from the terminal device may be transmitted to the communication device of the medicament device. Furthermore, data or information can be transmitted or transferred to the medicament device from terminal device. For example, a receipt of acknowledge can be transferred from the terminal device upon a succeeding transfer of information between the medicament device and terminal device to, for example, the medicament device, to a third party, such as a healthcare physician of the patient via the network computer system, to the other terminal device if two terminal devices are associated with one medicament device, or combinations thereof. This receipt of acknowledge can either be sent automatically upon receipt of the information or by a manual operation of the user of the terminal device.

According to one exemplifying embodiment of the present invention, the terminal device is connected to a network computer system via said wireless communication system. The network computer system may comprise a public access network computer system. The Internet is one suitable example of a public access network computer system, wherein the point of access thereto can be any suitable entry-point including an entry-point managed by an internet service provider. The public access network may also form part of a telecommunications system, which may itself be either a cellular system, an optical network or a wire network. Furthermore, the network computer system may comprises a private access network computer system of a medical centre or a medicament prescriber, for example a doctor's practice, of a patient using a medicament device of the system according to the present invention. The private access network system may for example comprise an Intranet or Ethernet which may for example, be maintained by a health service producer, a service provider, a pharmacy, or, as in this embodiment, a medical centre.

Accordingly, the terminal device can communicate with one or more third parties, such as healthcare providers, pharmacies, and other suppliers of healthcare products and services via the terminal device and the network computer system. For example, the user can be provided with instructions relating to the correct use of the device together with the recommended dosing amounts, dose intervals and treatment period by means of, for example, an MMS message, an SMS message, or the like. Consequently, a doctor can provide his patient with detailed medication information in an efficient and reliable manner, which, in addition, is easy to follow and comprehend for the patient.

In addition, information related to the medication of a patient can be transferred from the terminal device to the network computer system. As an example, the patient is able to communicate, for example, questions related to his medication to his doctor by means of, for example, an MMS message, an SMS message, or the like, either straight forward or by means of a predefined template. Thus, the communication between the patient and the doctor is facilitated and, in addition, efficient and reliable.

Furthermore, it is also advantageous, for example, when large amounts of medication data are to be collected from patients during research projects or clinical trial situations. In such cases, it is essential that the medication information relating to, for example, the patient medication compliance is correct. It is of frequent occurrence that patients participating in tests fail to take the proper medication or forget to take the medication without reporting this to the test staff of the research establishment, which may lead to incorrect test results. If the medication data relating to patient compliance instead is delivered or transferred directly to a database of the research establishment for from the medicament devices, the qualification and the registering of the medication data is significantly facilitated and render more effective and the risk of erroneous test results is greatly decreased.

According to embodiments of the present invention, the medicament device comprises a memory for storage of data related to the medication. The memory may comprise a non-volatile memory chip (e.g. an EEPROM or FLASH memory chip) which is capable of storing data, for example, when the transfer of data from the medicament device and the terminal device is disturbed, or during a predetermined period of time, for example during a clinical test.

Preferably, the data or the information is communicable between the communication device and the terminal device in encrypted form. Furthermore, the communication device may employ radiofrequency or optical signals. For example, the communication device can communicate with the terminal device using spread spectrum radiofrequency signals. A suitable spread spectrum protocol is the Bluetooth (trade mark) standard which employs rapid (e.g. 1600 times per second) hopping between plural frequencies (e.g. 79 different frequencies). The protocol may further employ multiple sending of data bits (e.g. sending in triplicate) to reduce interference. Hence, facilitating secure transmission of information packages.

According to embodiments of the present invention, a communication device can be arranged to communicate with at least two terminal devices.

Furthermore, a terminal device can be arranged to communicate with the communication devices of at least two different medicament devices.

As realized by the person skilled in the art, the method of the present invention, as well as preferred embodiments thereof, is suitable to realize or implement as a computer program or a computer readable medium, for instance within the contents of a terminal device, such as a cellular phone, an/or a medicament device, as well as within a public or a private access network computer system of a network computer system.

Further objects and advantages of the present invention will be discussed below by means of exemplifying embodiments.

### BRIEF DESCRIPTION OF THE DRAWINGS

Preferred embodiments of the invention will now be described in greater detail with reference to the accompanying drawings, in which

FIG. 1a schematically shows a block diagram of the relevant parts of a medicament device suitable for use in a method and a system according to the present invention;

FIG. 1b schematically shows an embodiment of the medicament device shown in FIG. 1a suitable for use in a method and a system according to the present invention;

FIG. 1c schematically shows another embodiment of the medicament device shown in FIG. 1a suitable for use in a method and a system according to the present invention;

FIG. 1d schematically shows yet another embodiment of the medicament device shown in FIG. 1a suitable for use in a method and a system according to the present invention;

FIG. 2 schematically shows an embodiment of a system for presenting and distributing information related to a medication of a patient according to the present invention;

FIG. 3 schematically shows a preferred embodiment of a method for presenting and distributing information related to a medication of a patient in the system disclosed in FIG. 2;

FIG. 4 schematically shows a second embodiment of a system for presenting and distributing information related to a medication of a patient according to the present invention; and

FIG. 5 schematically shows an embodiment of a method for presenting and distributing information related to a medication of a patient in the system disclosed in FIG. 4;

### DETAILED DESCRIPTION OF THE PREFERRED EMBODIMENT

In the following there will be disclosed preferred embodiments of the system and the method for presenting and distributing information related to a medication of a patient in accordance with the present invention.

Furthermore, a number of different medicament devices suitable for use in such a system and method will also be shown.

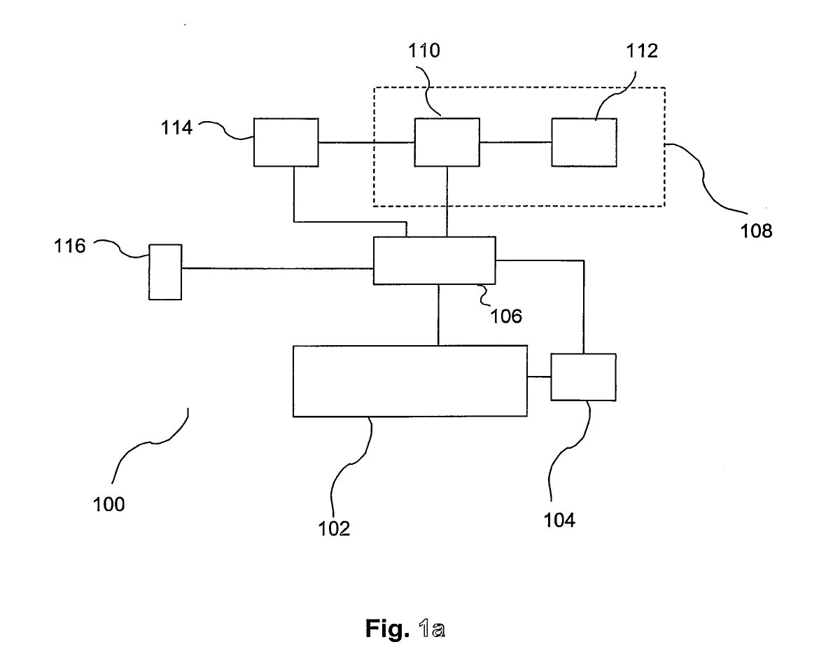

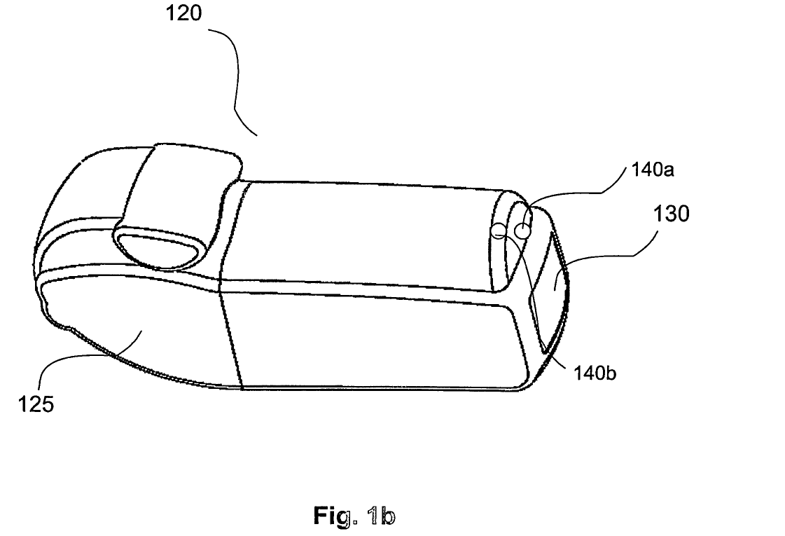

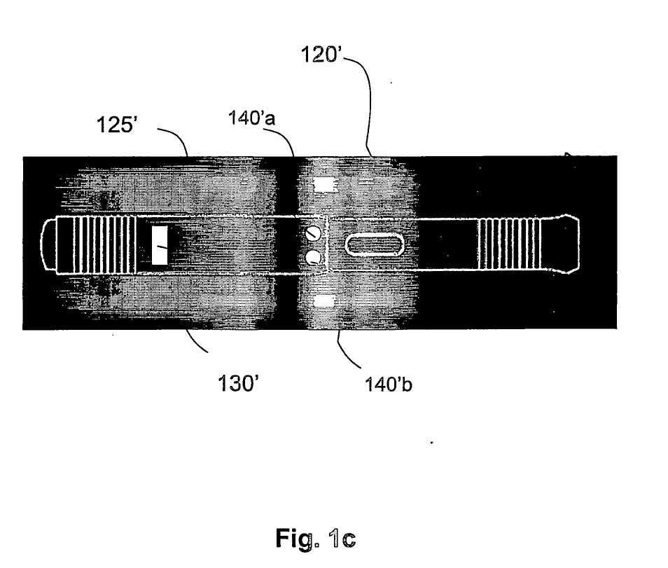

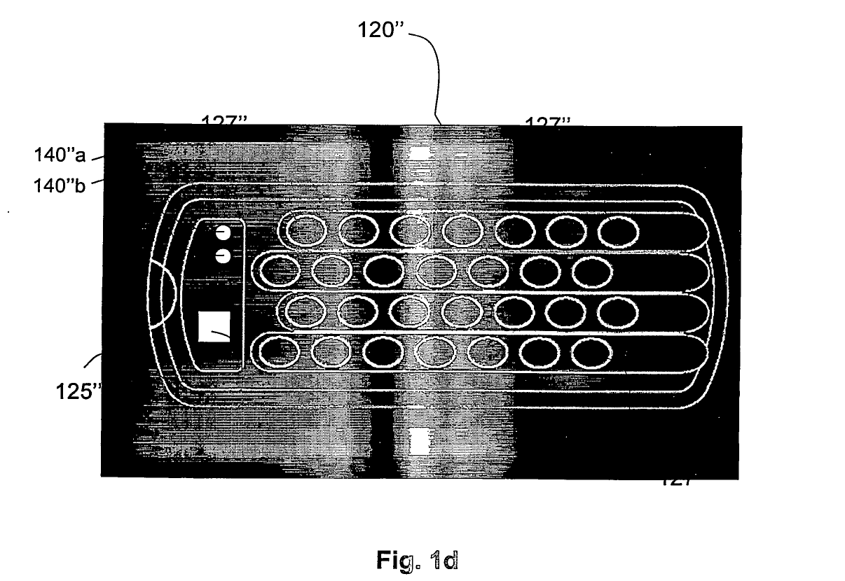

With reference first to FIG. 1a, a schematically block diagram of the relevant parts of a medicament device suitable for use in a method and a system according to the present invention is shown.

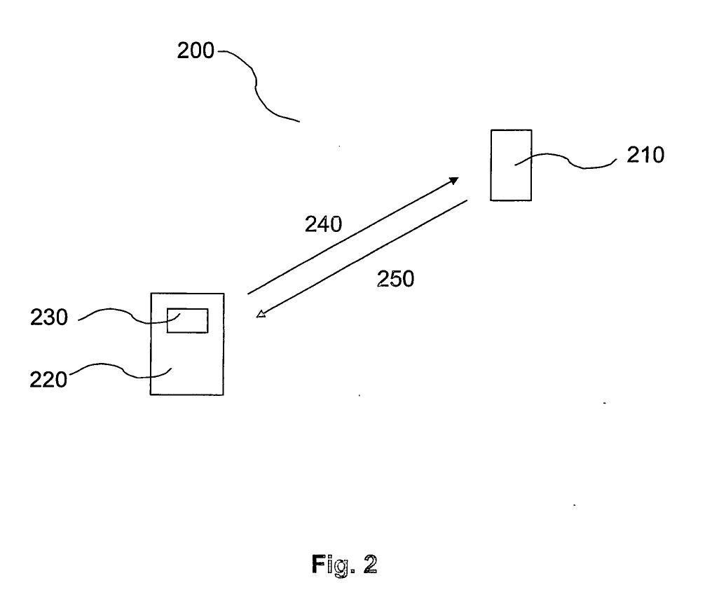

The medicament device 100 comprises a medicament containing means 102 arranged to hold a medicament, a dispensing mechanism 104 arranged for dispensing medicament from the medicament containing means 102, and means for obtaining information related to the medication 106. The means for obtaining information related to the medication 106 may be a sensor or a detector for detecting movements, for example, the activation of the dispensing mechanism. Other suitable techniques includes optical detectors, magnetic detectors or detectors using detection of capacitive effects. Moreover, a communication device 108 for wireless communication with a terminal device (see for example FIG. 2) to enable transfer of data to and from the medicament device 100 to the terminal device is arranged in the medicament device 100. The communication device 108 includes, inter alia, a transceiver 110 and an antenna 112 or equivalent for transmitting or receiving data.

Furthermore, the medicament device 100 comprises a memory 114 for storage of information related to the medication and medication events. The memory comprises a non-volatile memory chip (e.g. an EEPROM or FLASH memory chip) which is capable of storing the information, for example, when the transfer of data from the medicament device and the terminal device is disturbed, or during a predetermined period of time. Preferable, the medicament device is also provided with a power source (not shown) so as to powering the electrical components, e.g. the communication device 108. A suitable power source is a battery, clockwork energy store, solar cell, fuel cell or kinetics-driven cell. The power source may be arranged to be rechargeable or re-loadable. In addition, the medicament device 100 may be fitted with light elements 116, for example, one or more diodes.

With reference now to FIG. 1b, a schematic diagram of an embodiment of the medicament device shown in FIG. 1a suitable for use in a method and a system according to the present invention will be shown. The medicament device shown in FIG. 1b is an inhaler. Since use and the structure of an inhaler are well known to the man skilled in the art, for what reason only parts or components being relevant for the present invention will be described in detail.

The medicament device 120 comprises a housing 125 including a medicament containing means (see FIG. 1a) arranged to hold a medicament, a dispensing mechanism (see FIG. 1a) arranged for dispensing medicament from the medicament containing means, and means for obtaining information related to the medication (see FIG. 1a). The means for obtaining information related to the medication may be a sensor or a detector for detecting movements, for example, the activation of the dispensing mechanism. Other suitable techniques includes optical detectors, magnetic detectors or detectors using detection of capacitive effects. Moreover, the housing 125 comprises a communication device 130 for wireless communication with a terminal device (see for example FIG. 2) to enable transfer of data to and from the medicament device 120 to the terminal device. The communication device 130 includes, inter alia, a transceiver (see FIG. 1a) and an antenna (see FIG. 1a) or equivalent for transmitting or receiving data.

Preferably, the medicament device 120 comprises a memory (see FIG. 1a) for storage of information related to the medication and medication events. The memory comprises a non-volatile memory chip (e.g. an EEPROM or FLASH memory chip) which is capable of storing the information, for example, when the transfer of data from the medicament device and the terminal device is disturbed, or during a predetermined period of time. Preferable, the medicament device is also provided with a power source (see FIG. 1a) so as to powering the electrical components, e.g. the communication device 130. A suitable power source is a battery, clockwork energy store, solar cell, fuel cell or kinetics-driven cell. The power source may be arranged to be rechargeable or re-loadable. The medicament device can be arranged to send status indicating messages to a terminal device indicating when the battery capacity, for example, has reached a certain predetermined capacity level, thereby giving the user a warning in advance of that the battery capacity is about to run out. This can be executed by means of a notification in the form of an audio signal or an SMS message.

Moreover, the medicament device 120 is fitted with light elements, for example, one or more diodes. In the embodiment shown in FIG. 1a, the medicament device 120 includes a first diode 140a, which in this embodiment is red, and a second diode 140b, which in this embodiment is red. The red diode 140a can be arranged to twinkle or to give a fixed light when the battery capacity has reached the certain predetermined capacity level, thereby giving the user a warning in advance that the battery capacity is about to run out. Correspondingly, the second red diode 140b can be arranged to twinkle or to give a fixed light if a message from the medicament device to a terminal device for some reason could not be transferred correctly. The man skilled in the art easily realizes that there are a plurality of alternatives and conceivable modifications to this described configuration and to the described functions of the light elements.

Preferably, the communication device 130 communicates with the terminal device using radiofrequency signals, for example, spread spectrum radiofrequency signals. A suitable spread spectrum protocol is the Bluetooth (trade mark) standard which employs rapid (e.g. 1600 times per second) hopping between plural frequencies (e.g. 79 different frequencies). The protocol may further employ multiple sending of data bits (e.g. sending in triplicate) to reduce interference. Of course, the communication device 130 can use other ways for communicate with a terminal device, for example, optical signals. Moreover, the communication device 130 may be arranged for two-way transfer of information. Accordingly, a terminal device can, in addition to receive information from the communication device 130, transfer information to the communication device 130.

Referring now to FIG. 1c, a schematic diagram of a another embodiment of the medicament device shown in FIG. 1a suitable for use in a method and a system according to the present invention will be shown. The medicament device shown in FIG. 1c is an injector. Since use and the structure of an injector are well known to the man skilled in the art, for what reason only parts or components being relevant for the present invention will be described in detail.

The medicament device 120′ comprises a housing 125′ including a medicament containing means (see FIG. 1a) arranged to hold a medicament, a dispensing mechanism (see FIG. 1a) arranged for dispensing medicament from the medicament containing means, and means for obtaining information related to the medication (see FIG. 1a). The means for obtaining information related to the medication may be a sensor or a detector for detecting movements, for example, the activation of the dispensing mechanism. Other suitable techniques includes optical detectors, magnetic detectors or detectors using detection of capacitive effects. Moreover, the housing 125′ comprises a communication device 130′, as described with reference to, for example, FIG. 1a, for wireless communication with a the terminal device (see for example FIG. 2). In addition, the medicament device 120′ comprises a memory (see FIG. 1a), power source (see FIG. 1a), and light elements 140′a, 140′b as described with reference to FIGS. 1a and 1b.

Turning now to FIG. 1d, a schematic diagram of yet another embodiment of the medicament device shown in FIG. 1a suitable for use in a method and a system according to the present invention will be shown. The embodiment shown in FIG. 1d is a tablet dispenser. Since use and the structure of a tablet dispenser are well known to the man skilled in the art, for what reason only parts or components being relevant for the present invention will be described in detail.

The medicament device 120″ comprises a housing 125″ including a medicament containing means, which in this embodiment includes a number of medicament compartments 127″ arranged for holding medicaments, a dispensing mechanism (see FIG. 1a) arranged for dispensing medicament from the medicament compartments 127″, and means for obtaining information related to the medication (see FIG. 1a). The means for obtaining information related to the medication may be a sensor or a detector for detecting movements, for example, the activation of the dispensing mechanism. Other suitable techniques includes optical detectors, magnetic detectors or detectors using detection of capacitive effects. Moreover, the housing 125″ comprises a communication device 130″, as described with reference to, for example, FIG. 1a, for wireless communication with a terminal device (see for example FIG. 2). In addition, the medicament device 120″ comprises a memory (see FIG. 1a), power source (see FIG. 1a), and light elements 140″a, 140″b, as described with reference to FIGS. 1a and 1b.

With reference now to FIG. 2, a schematic diagram of a first embodiment of a system for presenting and distributing information related to a medication of a patient in accordance with the present invention will be shown. The system 200 comprises a terminal device 210 capable of communicating with a wireless communication system (not shown) and comprising medication compliance information functions. Preferably, the terminal device 210 is a telecommunication device, more preferably a cellular phone. Alternatively, the terminal device can be a PDA.

Furthermore, the system 200 includes a medicament device 220 adapted for the delivery of a medicament or the administration of a drug to a patient (not shown) as described with reference to any one of the FIGS. 1a, 1b, 1c, and 1d. Of course, there are other conceivable types of medicament devices is suitable for use in the system according to the present invention such as Metering Dose Inhalers (MDI), Dry Powder Inhalers (DPI), nebulizers portable or stationary, as well as inhalers adapted for, for example, rectally dispensing of the medicament, other types of tablet dispensers or other types of injectors.

Preferably, the communication device 230 communicates with the terminal device 210 using radiofrequency signals, for example, spread spectrum radiofrequency signals. As indicated above, a suitable spread spectrum protocol is the Bluetooth. The protocol may further employ multiple sending of data bits (e.g. sending in triplicate) to reduce interference. Of course, the communication device 230 can use other ways for communicating with the terminal device, for example, optical signals. Moreover, the communication device 230 may be arranged for two-way transfer of information. This is represented in FIG. 1 by the arrows 240 and 250, respectively. Accordingly, the terminal device 210 can, in addition to receive information from the communication device 230, transfer information to the communication device 230, for example, upon a succeeding transfer of information from the medicament device 230, a receipt of acknowledge.

In an alternative embodiment, the system 200 may comprise two terminal devices 210 comprising medication compliance information functions connected to one medicament device 230. Thereby, each terminal device 210 can be provided with the same information simultaneously or almost simultaneously. This improves the medical compliance, for example, in cases when patients suffering from senile dementia. The patient can be provided with one of the terminal devices and a relative or a member of a nursing staff attending the patient with the other, and, for instance if the patient forgets taking the medication the relative or the member of the nursing staff will be notified as well as the patient. This communication can be performed either serially, i.e. from the medicament device 230 the first terminal device and further to the second, or in parallel, i.e. from the medicament device 230 to both terminal devices simultaneously.

Of course there are other conceivable embodiments of the system according to the present invention. For example, an arbitrary number of medicament devices may be connected to a specific terminal device, each medicament device being associated with one user or patient. If two medicament devices are included in the system and connected to one terminal device a member of a nursing staff may, thereby, readily be notified of the medical compliance of two different patients or more.

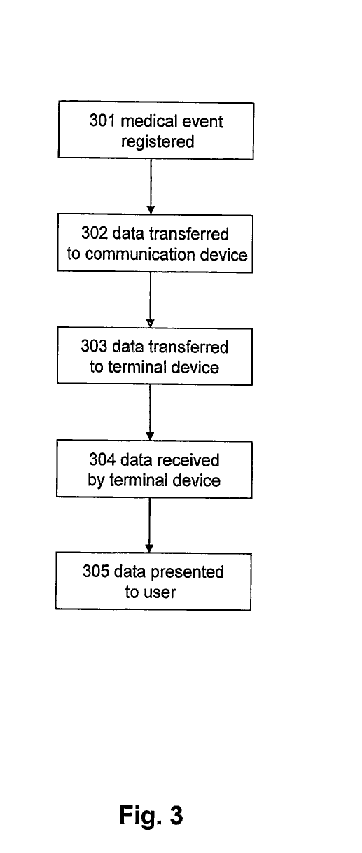

Referring now to FIG. 3, a preferred method of presenting information related to a medication of a patient in a system according to the first embodiment of the present invention will be described. At step 301, a medication event is observed and/or registered by the means for obtaining information related to the medication. To this end, the means for obtaining information constantly monitors the dispensing of medicaments in order to register events, or the absence of events, related to the dispensing of the medicament. A medication event may be, for example, that the patient using the medicament device 220 takes a wrong amount of the medicament or forgets to take the medication. As an alternative, the information or data related to the medication event can be stored in the memory of the medicament device 220. For example, the information related to a predetermined number of events may be stored, or the information related to all events occurring during a predetermined period of time may be stored. Alternatively, the information related to the medication events may be stored only if the communication device 230 for some reason was not able to transfer the information related to the event or events.

At step 302, the information is transferred to the communication device 230. Then, at step 303, the information is wirelessly transferred or sent to the terminal device 210. In case of disturbances or the like of the information transfer or if a link could not be established between the communication device 230 and the terminal device 210 preventing the communication device 230 from transferring the information to the terminal device 210, the information may be stored in the memory of the medicament device 220, as mentioned above. Thereafter, when a connection is established, the information can be sent or transferred to the terminal device 210. However, at step 304, the information is received by the terminal device 210, and, if necessary, processed by the processing means of the terminal device 210. At step 305, the information is presented to the user of the terminal device 210 by using the in-built functionality of the terminal device 210, which functionality includes the medication compliance information functions. The user of the terminal device 210 may of course be the patient using the medicament device 220, but may also be a relative or a member of a nursing staff. Accordingly, it is possible to, for example, remind and/or notify the user that he or she has forgotten to take the medication or that he or she has not taken the proper medication (or, if the user is not the patient, that the patient has forgotten to take the medication or has not taken the proper medication) utilizing the in-built functionality of the terminal device 210. Preferably, the user is notified by means for notifying selected from the group consisting of means for providing audible signals, visualizing means, vibration means, and light means. The means for proving audible signals may be, for example, a speaker phone of a cellular phone, which is able to notify the user by producing an audible signal. In order to increase the medication compliance, the user, which often is the patient, can select a specific signal, for example, a characteristic tune or sound. The visualizing means may be, for example, a display comprising a screen such as an LED or LCD. If a cellular phone is utilized the user may be notified, for example, by an evident symbol, a message, an instructive sequence of pictures, or a film sequence shown on the display. Preferably, the user may be notified by means of, for example, an SMS message, an MMS message, or the like. If a cellular phone is used the vibration means may be, for example, a vibration unit. The user is notified by vibrations from the unit. This is advantageous in noisy environments such as a subway or in a restaurant. The light means may be, for example, a diode of a cellular phone.

Of course, there are a plurality of other conceivable methods and the above embodiment should be considered only as an exemplification. As realized by the person skilled in the art, the above method, as well as preferred embodiments thereof, is suitable to realize or implement as a computer program or a computer readable medium.

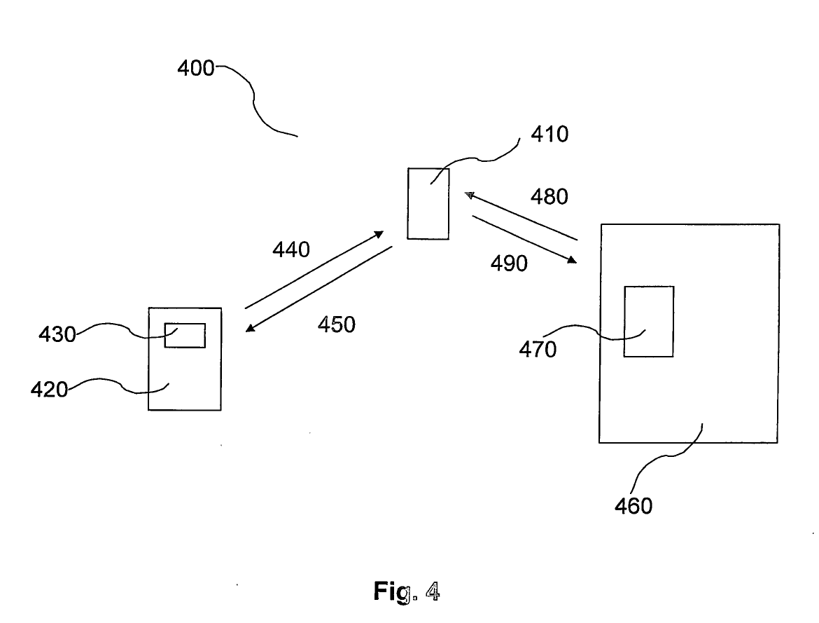

With reference now to FIG. 4, a schematic diagram of a second embodiment of the system according to the present invention will be shown. The system 400 comprises a terminal device 410 as described above and a medicament device 420 adapted for the delivery of a medicament to a patient (not shown) as described with reference to any one of the FIGS. 1a, 1b, 1c, or 1d. The medicament device 420 includes a communication device 430, which is able to communicate with the terminal device 410 using radiofrequency signals, for example, spread spectrum radiofrequency signals. As indicated above, a suitable spread spectrum protocol is the Bluetooth. Moreover, the communication device 430 may be arranged for two-way transfer of information, which is represented by the arrows 440 and 450, respectively. Accordingly, the terminal device 410 can, in addition to receive information from the communication device 430, transfer information to the communication device 430.

Furthermore, the terminal device 410 is connected to a network computer system 460 via a wireless communication system (not shown) for two-way transfer of data between the terminal device 410 and the network computer system 460, as represented by the arrows 480 and 490. In this embodiment, the network computer system 460 comprises a public access network computer system. The Internet is one suitable example of a public access network computer system, wherein the point of access thereto can be any suitable entry-point including an entry-point managed by an internet service provider. The public access network may also form part of a telecommunications system, which may itself be either a cellular system or an optical network. In this embodiment, the network computer system 460 comprises a private access network computer system 470 of a medical centre or a medicament prescriber, for example a doctor's practice, of the patient using the medicament device 420. The private access network system 470 may for example comprise an Intranet or Ethernet which may for example, be maintained by a health service producer, a service provider, a pharmacy, or, as in this embodiment, a medical centre. Accordingly, the terminal device 410 can communicate with one or more third parties, such as medical centres, healthcare providers, and other suppliers of healthcare products and services via the wireless communication system and the network computer system 460. In this embodiment, the patient, accordingly, is able to communicate with his or her doctor using the terminal device 410, for instance, by means of an SMS message, an MMS message, or the like. Furthermore, the doctor is also able to communicate with the patient, for example, by means of an SMS message, an MMS message, or the like.

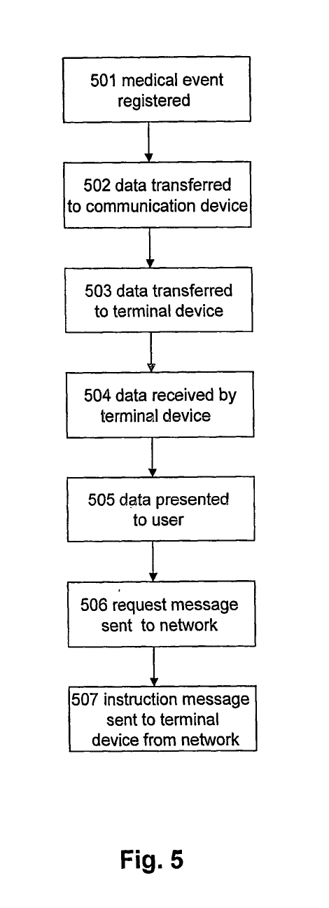

Referring now to FIG. 5, an embodiment of a method of presenting information related to a medication of a patient in a system according to the second embodiment of the present invention will be described. At step 501, a medication event is observed and/or registered by the means for obtaining information related to the medication. To this end, the means for obtaining information constantly monitors the dispensing of medicaments in order to register events related to the dispensing. A medication event may be for example, that the patient using the medicament device 420 has forgotten to take the medication or that he or she has not taken the proper medication.

Then, as an alternative, the information or data related to the medication event can be stored in the memory of the medicament device 420. For example, the information related to a predetermined number of events may be stored, or the information related to all events occurring during a predetermined period of time may be stored. Alternatively, the information related to medication events may be stored only if the communication device 430, for some reason was not able to transfer the information related to the event.

At step 502, the information is transferred to the communication device 410. Thereafter, at step 504, the information is wirelessly transferred or sent to the terminal device 410. In case of disturbances or the like of the information transfer or if a link could not be established between the communication device 430 and the terminal device 410 preventing the communication device 430 from transferring the information to the terminal device 410, the information may be stored in the memory of the medicament device, as mentioned above. Thereafter, when a connection is established, the information can be sent or transferred to the terminal device 410. At step 505, the information is received by the terminal device 410, and, if necessary, processed by the processing means (not shown) of the terminal device 410. Subsequently, at step 506, the information is presented, as described above, to the user of the terminal device 410 by using the in-built functionality of the terminal device 410, which functionality comprises the medication compliance information functions. At step 507, a message is sent from the terminal device 410 to the private access network computer system 470 of the medical centre of the patient containing information that a certain event has occurred. This can be achieved by means of an automatic service where the message is sent directly and automatically upon receipt of the information related to the event. Alternatively, the patient may, upon notification of the event, create a message, for example in the form of an SMS message, using the in-built functionality of the terminal device 410 and send it to the private access network computer system 470 of the medical centre containing a request for information about how to react upon the occurred event. For example, which actions that should be taken when the patient has forgotten to take the medication. Upon receipt of the request message, the private access network computer system 470 of the medical centre may send an instruction message containing information describing which actions the patient should take. This message can, for example, be in the form of an SMS message, an MMS message, or the like.

Furthermore, the user or the patient can be provided with instructions relating to the correct use of the device together with the recommended dosing amounts, dose intervals and treatment period by means of, for example, an MMS message, an SMS message, or the like. Consequently, a doctor can provide his patient with detailed medication information in an efficient and reliable manner which, in addition, is easy to follow and comprehend for the patient.

Furthermore, when, for example, large amounts of medication data are to be collected from patients during research projects or clinical trial situations, it is essential that the medication information relating to, for example, the patient compliance is correct. It is of frequent occurrence that patients participating in tests fail to take the proper medication or forget to take the medication without reporting this to the test staff of the research establishment, which may lead to incorrect test results. If the medication data, i.e. information related to medication events, related to the compliance of the patient participating in the test is delivered or transferred directly to a database of the research establishment from the medicament device using the terminal device, the qualification, the collection, and the registering of the medication data are significantly facilitated and render more effective and the risk of erroneous test results is greatly decreased.

Although specific embodiments have been shown and described herein for purposes of illustration and exemplification, it is understood by those of ordinary skill in the art that the specific embodiments shown and described may be substituted for a wide variety of alternative and/or equivalent implementations without departing from the scope of the present invention. Those of ordinary skill in the art will readily appreciate that the methods of the present invention could be implemented in a wide variety of embodiments, including hardware and software implementations, or combinations thereof. This application is intended to cover any adaptations or variations of the preferred embodiments discussed herein. Consequently, the present invention is defined by the wording of the appended claims and equivalents thereof.

1. A system for presenting and distributing information related to a medication of a patient comprising a terminal device communicating with a wireless communication system, and a medicament device including a medicament containing means arranged to hold a medicament; a dispensing mechanism arranged for dispensing medicament from the medicament containing means; means for obtaining information related to the medication of the patient; and a communication device for wireless communication to enable transmission of data related to said information from the medicament device to a terminal device, wherein said terminal device comprises means for receiving said data from said communication device and means for providing the user with a notification based on said information, and which device is arranged to present information related to the medication of the patient to a user.

2. The system according to claim 1, wherein the terminal device is arranged to present information obtained from the medicament device by said means for obtaining information related to the medication and transferred to the terminal device from the medicament device via the communication device.

3. The system according to claim 1, wherein the means for providing the user with a notification is selected from the group consisting of means for providing audible signals, visualizing means, vibration means, and light means.

4. The system according to claim 1, wherein the communication device is arranged for two-way transfer of data between the terminal device and the medicament device and the terminal device comprises means for transmitting data from the terminal device to the communication device.

5. The system according to claim 1, wherein the terminal device is connected to a network computer system via said wireless communication system, thereby enabling a transfer of information related to the medication of the patient between the network computer system and the terminal device.

6. The system according to claim 1, wherein the means for obtaining information related to the dispensing is arranged to monitor the dispensing of medicament and is connected to the communication device to transfer data related to dispensing events.

7. The system according to claim 1, wherein the medicament device comprises a memory for storage of data related to the medication.

8. The system according to claim 1, wherein the terminal device is a cellular phone.

9. The system according to claim 1, wherein the data is communicable between the communication device and the terminal device in encrypted form.

10. The system according to claim 1, wherein the communication device employs radiofrequency or optical signals.

11. The system according to claim 1, wherein the communication device communicates with the terminal device using spread spectrum radiofrequency signals.

12. The system according to claim 1, wherein the medicament device is arranged to transfer data related to the medication of a patient to at least two terminal devices.

13. The system according to claim 1, wherein said means for receiving data is arranged to receive information from at least two communication devices.

14. A method of presenting information related to a medication of a patient in a system comprising a terminal device communicating with a wireless communication system, and a medicament device including a medicament containing means arranged to hold a medicament; a dispensing mechanism arranged for dispensing medicament from the medicament containing means; means for obtaining information related to the medication of the patient; and a communication device for wireless communication to enable transfer of data related to said information from the medicament device to a terminal device, the method comprising the step of utilizing the terminal device to present information related to the medication of the patient to a user; and notifying the user of an event related to the medication using the terminal device.

15. The method according to claim 14, further comprising the steps of obtaining information from the medicament device by said means for obtaining information related to the medication; and transmitting said data to the terminal device from the medicament device via the communication device.

16. The method according to claim 14, wherein the step of notifying the user comprises the step of notifying the user by means of means for notifying selected from the group consisting of means for providing audible signals, visualizing means, vibration means, and light means.

17. The method according to claim 14, wherein the step of notifying the user comprises the step of notifying the user by means of an SMS message, an MMS message, or the like.

18. The method according to claim 14, wherein the communication device is arranged for two-way transfer of data between the terminal device and the medicament device, the method further comprising the step of transmitting data from the terminal device to the communication device.

19. The method according to claim 14, wherein the terminal device is connected to a network computer system via said wireless communication system, the method further comprising the step of transmitting information related to the medication of the patient to the network computer system from the terminal device or from the network computer system to the terminal device.

20. The method according to claim 14, wherein means for obtaining information related to the dispensing is arranged to monitor the dispensing of medicament and is connected to the communication device to transfer data related to the dispensing, the method further comprising the steps of monitoring the dispensing of medicament; and transmitting information related to the dispensing from the terminal device to the communication device.

21. The method according to claim 14, further comprising the step of storing data related to the medication.

22. The method according to claim 14, wherein the data is communicable between the communication device and the terminal device in encrypted form.

23. The method according to claim 14, wherein the communication device employs radiofrequency or optical signals.

24. The method according to claim 14, wherein the communication device communicates with the terminal device using spread spectrum radiofrequency signals.

25. The method according to claim 14, wherein the communication device communicates with at least two terminal devices.

26. The method according to claim 14, wherein the terminal device communicates with a least two communication devices.

27. Use of a terminal device in a system for the delivery of medicament to present information related to the medication of a patient, which system comprises a medicament device including a medicament containing means arranged to hold a medicament; a dispensing mechanism arranged for dispensing medicament from the medicament containing means; means for obtaining information related to the medication of the patient; and a communication device for wireless communication to enable transfer of data from the medicament device to the terminal device, to present information related to the medication of the patient to a user.

28. Computer readable medium comprising instructions for bringing a programmable device to perform the method according to claim 14.
